(19) United States
(12) Patent Application Publication
(10) Pub. No.: US 2008/0171762 A1
(43) Pub. Date: Jul. 17, 2008

(54) Treatment of pain with naloxone

(76) Inventor: David M. Ockert, New York, NY (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/653,582

(22) Filed: Jan. 16, 2007

(51) Int. Cl. A61K 31/485; A61P 19/00; A61P 21/00

(52) U.S. Cl. 514/282

(57) ABSTRACT

A method of effectively treating nociceptive pain involves administering to a human or animal in need of relief from nociceptive pain a therapeutically effective amount of naloxone intranasally, intravenously, by inhalation, transdermally, or orally. Pain associated with fibromyalgia, arthritis, surgical procedures, etc. can be effectively treated.

### FIELD OF THE INVENTION

This invention relates to the treatment of pain, and more particularly to the treatment of pain with naloxone.

### BACKGROUND OF THE INVENTION

The inventor has been issued U.S. Pat. No. 5,376,662 relating to the daily administration to a patient in need of relief from nerve injury induced pain of from about 0.4 milligrams to about 2.0 milligrams of the opiate-receptor antagonist naloxone. Prior to the inventor's discovery that naloxone can be utilized for the treatment of nerve injury induced pain, naloxone had been used for the treatment of respiratory depression secondary to opiate overdose, and to diagnose opiate dependence.

U.S. patent application Publication No. 2006/0083691 authored by Daniel P. Wermeling, discloses intranasal administration of a composition comprising a therapeutically effective amount of an opioid as part of a method for treating a mammal suffering from pain. Opioids that are alleged to be suitable for use in the invention include morphine, apomorphine, dihydromorphine, diacetylmorphine, hydromorphone, hydrocodone, oxymorphone, lavorphanol, levallorphan, levophenacylmorphan, norlevorphanol, nalorphine, nalbuphine, buprenorphine, butorphanol, naloxone, methadone, oxycodone, naltrexone, nalmexone, oxilorphan, cyclorphan, ketobemidone, fentanyl, sufentanil, alfentanyl, and combinations thereof. The compositions disclosed by Wermeling are said to elicit analgesia or an analgesic response to relieve or alleviate pain in a subject, with specifically disclosed diseases and/or conditions that cause pain including cancer, arthritis, neurological diseases, heat attacks, trauma, childbirth, migraines, or surgery, dental procedures, etc. There is not any guidance as to which of the numerous disclosed opioids is useful for treating pain associated with a particular one of the disclosed diseases and/or conditions that cause pain. Presumably, one of ordinary skill in the art may refer to the literature for guidance as to which opioid is useful for treating a specific type of pain.

It is believed that the only known use of naloxone for effectively treating pain is the treatment disclosed in Dr. Ockert's U.S. Pat. No. 5,376,662, which is limited to the treatment of pain induced by nerve injury.

### SUMMARY OF THE INVENTION

It has been surprisingly discovered that naloxone is effective in the treatment of nociceptive pain and disease induced neuropathic pain.

In one aspect of the invention, there is provided an effective method of treating a human or animal patient suffering from nociceptive pain and/or disease induced neuropathic pain comprising administering to the patient a therapeutically effective amount of naloxone.

These and other features, advantages and objects of the present invention will be further understood and appreciated by those skilled in the art by reference to the following specification and claims.

### DETAILED DESCRIPTION OF PREFERRED EMBODIMENTS

The method of the present invention consists of the administration to a human or animal patient suffering from nociceptive pain and/or disease induced neuropathic pain a therapeutically effective amount of naloxone.

Nociceptive pain, as used herein and as defined in the literature, is pain that results from tissue damage, and wherein there is not any substantial nerve damage. Instead, intact neurons report the tissue damage, and pain is experienced. Nociceptive pain can be cutaneous pain, somatic pain or visceral pain. Nociceptive pain can be experienced as sharp, dull or aching. In addition, nociceptive pain can be either acute or chronic. Cutaneous pain is pain caused by injury to the skin or superficial tissues. Cutaneous nociceptors terminate just below the skin, and due to a large number of nerve endings per unit area, produce a well-defined localized pain of relatively short duration. Examples of injuries that produce cutaneous pain include paper cuts, minor cuts, minor (first degree) burns and lacerations. Somatic pain may originate from ligaments, tendons, bones, and blood vessels. It is detected with somatic nociceptors. The scarcity of somatic nociceptors in these areas produces a dull, poorly-localized pain of longer duration than cutaneous pain. Examples include sprains and broken bones. Visceral pain originates from viscera (i.e., organs of the body). Visceral nociceptors are located within body organs and internal cavities. The even greater scarcity of nociceptors in these areas produces pain that is usually more aching and of a longer duration than somatic pain. Visceral pain is extremely difficult to localize and several injuries to visceral tissue exhibit a pain in which the sensation is localized to an area completely unrelated to the site of injury. This is known as referred visceral pain. Myocardial ischaemia (the loss of blood flow to a part of the heart muscle tissue) is possibly the best known example of referred visceral pain. The sensation can occur in the upper chest as a restricted feeling, or as an ache in the left shoulder, arm or even hand. Referred visceral pain can be explained by the findings that pain receptors in the viscera also excite spinal cord neurons that are excited by cutaneous tissue. Since the brain normally associates firing of the spinal cord neurons with stimulation of somatic tissues in skin or muscle, pain signals arising from the viscera are interpreted by the brain as originating from the skin.

Encompassed within the category of nociceptive pain are fibromyalgia (i.e., chronic pain in muscles and soft tissue surrounding joints), arthritis, and other inflammatory diseases of ligaments and tendons. Other types of nociceptive pain that may be treated with naloxone in accordance with this invention include phantom limb pain, complex regional pain syndrome and post-operative pain.

Complex regional pain syndrome is a chronic pain caused by a physical injury, in which the pain is typically of longer duration and greater intensity than what would be expected from the injury.

Other forms of nociceptive pain and/or disease induced pain that are not associated with substantial nerve damage and which are effectively treated in accordance with the method of the invention include anoxic, Raynauds, myofacial, autoimmune, ischemic, as well as certain types of nociceptive pain induced by neuropathic processes, diffuse non-organic pain, non-organic back pain, trigeminal pain, connective tissue diseases, diabetic neuropathy, shingles pain syndrome, fibromyalgia, ligament sprain, arthritis, headache, migraine pain, tendon pain, ligament pain, arachnoiditis-induced pain, chronic pain, endometriosis, and nerve pain associated with diabetes or shingles.

Disease induced, neuropathic pain refers to neuropathic pain caused by infections or autoimmune disorders that affect nerve tissue, idiopathic neuropathies (those without a known cause), and systemic diseases that cause peripheral neuropathy. Diseases that can cause peripheral neuropathy include multiple sclerosis, kidney disorders that produce substances damaging to nerve tissue, hormonal imbalances, dietary deficiencies of one or more vitamins essential to healthy nerve function (e.g., vitamins E, B1, B6, B12, niacin and thiamin), vascular damage and blood diseases that decrease oxygen supply to peripheral nerves, connective tissue disorders and chronic inflammation that cause nerve damage, cancers and tumors that exert damaging pressure on nerve fibers, and toxins that cause peripheral nerve damage. Infections and autoimmune disorders that can cause peripheral neuropathy include viruses and bacteria that can attack nerve tissues (herpes, varicella-zoster, Epstein-Barr virus, cytomegalovirus, and herpes simplex), human immunodeficiency virus (HIV), Lyme disease, diphtheria, and leprosy.

Naloxone may be administered in the treatment of nociceptive pain on a daily or as needed basis, with a suitable daily dosage being from about 0.4 milligrams to about 4.0 milligrams. Multiple daily doses may also be administered in appropriate correspondingly smaller amounts. The most suitable doses are dependent on the type and location of the source of nociceptive pain, and the physical characteristics of the patient (e.g., age, weight, etc.). Appropriate doses may be determined by utilizing routine experimental techniques, and/or by trial and error within the perimeters provided above.

In accordance with certain embodiments of the invention, naloxone may be administered along with other pharmacological and/or non-pharmacological treatments for patients experiencing nociceptive pain and/or disease induced neuropathic pain. Such treatments will vary depending upon a particular patient's diagnosis. However, the typical course of treatment preferably includes some adjunctive treatment (pharmacological and/or non-pharmacological) for the duration of the period (e.g., six months) during which the patient is being treated with naloxone. Examples of adjunctive treatments include anti-inflammatory agents, serotonin specific reuptake inhibitors (SSRIs) and/or physical therapy. The methods of treatment of this invention permit patients, as the symptoms of their nociceptive pain and/or disease induced neuropathic pain subside, to more easily engage in such non-pharmacological treatments as physical therapy.

Relief of pain occurs in about one hour and is continually enhanced over approximately the next three weeks. At the end of the treatment period (typically about six months), the patient will be discontinued from naloxone administrations, with some patients remaining pain free for an extended period of time after discontinuation of treatment with naloxone.

The administration of naloxone to treat nociceptive pain and/or disease induced neuropathic pain may be achieved intranasally, intravenously, intramuscularly, by inhalation, transdermally, and/or orally (in immediate release, sustained release, or other modified release form).

Relief of nociceptive pain and/or disease induced neuropathic pain typically occurs within about one hour for any of the routes of administration. However, the onset of relief is generally hastened by intravenous or intramuscular injection at or near the source of pain. Inhalation and intranasal administration also provide relatively quick relief and has the advantage of greater patient acceptance and compliance as compared with intravenous and/or intramuscular injection.

The above description is considered that of the preferred embodiments only. Modifications of the invention will occur to those skilled in the art and to those who make or use the invention. Therefore, it is understood that the embodiments described above are merely for illustrative purposes and not intended to limit the scope of the invention, which is defined by the following claims as interpreted according to the principles of patent law, including the doctrine of equivalents.

The invention claimed is:

1. A method of treating nociceptive pain and/or disease induced neuropathic pain comprising administering to a human or animal patient in need of relief from nociceptive pain and/or disease induced neuropathic pain a therapeutically effective amount of naloxone.

2. The method of claim 1, wherein naloxone is administered intranasally.

3. The method of claim 1, wherein naloxone is administered intravenously.

4. The method of claim 1, wherein naloxone is administered by inhalation.

5. The method of claim 1, wherein naloxone is administered transdermally.

6. The method of claim 1, wherein naloxone is administered orally.

7. The method of claim 1, wherein naloxone is administered orally in a sustained release form.

8. The method of claim 1, in which the nociceptive pain is attributed to cutaneous tissue damage.

9. The method of claim 1, in which the pain is nociceptive pain that is attributed to fibromyalgia.

10. The method of claim 1, in which the pain is nociceptive pain that is attributed to arthritis.

11. The method of claim 1, in which the pain is nociceptive pain that is attributed to post-operative pain.

12. The method of claim 1, in which the pain is nociceptive pain that is attributed to phantom limb pain.

13. The method of claim 1, in which the pain is nociceptive pain that is attributed to a complex regional pain syndrome.

14. The method of claim 1, in which the pain is disease induced pain.

15. The method of claim 1, in which the pain is induced by idiopathic neuropathies.

16. The method of claim 1, in which the pain is caused by a systemic disease.

17. The method of claim 1, in which the pain is caused by an infection.

18. The method of claim 1, in which the pain is caused by an autoimmune disorder.

19. The method of claim 1, in which the pain is a nociceptive pain or disease induced pain, which pain is not associated with substantial nerve damage, and which pain is at least one of Raynauds, anoxic, myofacial, ischemic, headache, diffuse non-organic, non-organic back pain, trigeminal pain, connective tissue disease, diabetic neuropathy, shingles, ligament sprain, migraine, tendon, ligament, arachnoiditis-induced, chronic, and endometriosis pain.
